(19) United States
(12) Patent Application Publication
(10) Pub. No.: US 2015/0089785 A1
(43) Pub. Date: Apr. 2, 2015

(54) METHOD OF ATTACHING AN ELEMENT TO A DRIVE SHAFT

(71) Applicant: Cardiovascular Systems, Inc., St. Paul, MN (US)

(72) Inventors: Victor Blackledge, Cologne, MN (US); Benjamin Haselman, St. Paul, MN (US); Joseph Higgins, Minnetonka, MN (US); Joseph Bahoora, Medina, MN (US); Nick Ellering, Crystal, MN (US)

(73) Assignee: Cardiovascular Systems, Inc., St. Paul, MN (US)

(21) Appl. No.: 14/041,559

(22) Filed: Sep. 30, 2013

(51) Int. Cl. A61B 17/3207; B23K 31/02

(52) U.S. Cl. CPC A61B 17/3207; B23K 31/025
USPC 29/426.1; 29/428; 228/164

(57) ABSTRACT

A method for attaching a head to a drive shaft may include winding a wire to create a drive shaft, selecting a head, forming a tapered tip on a distal end of the drive shaft, placing the head on the tapered tip such that a distal end of the tapered tip extends from a distal end of the head, engaging the distal end of the tapered tip and turning down the drive shaft to create a turned down portion, advancing the head proximally over the turned down portion, and disengaging the distal end of the tapered tip wherein the drive shaft frictionally engages the head.

### BACKGROUND OF THE INVENTION

1. Field of the Invention

The invention relates to devices and methods for removing tissue from body passageways, such as removal of atherosclerotic plaque from arteries, utilizing a high-speed rotational atherectomy device. More particularly, the present invention relates to attachment of heads that may be used with an atherectomy device.

2. Description of the Related Art

A variety of techniques and instruments have been developed for use in the removal or repair of tissue in arteries and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patency of the opened artery. For non-calcified lesions, balloon angioplasty most often is used alone to open the artery, and stents often are placed to maintain patency of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patency of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. The disclosure of U.S. Pat. No. 5,314,438 is hereby incorporated by reference in its entirety.

U.S. Pat. No. 5,681,336 (Clement) provides an eccentric tissue removing burr with a coating of abrasive particles secured to a portion of its outer surface by a suitable binding material. This construction is limited, however because, as Clement explains at Col. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, in this prior art device, the center of mass offset from the rotational axis of the drive shaft would result in development of significant centrifugal force, exerting too much pressure on the wall of the artery and creating too much heat and excessively large particles.

U.S. Pat. No. 5,584,843 (Wulfman) discloses one or more ellipsoidal burrs or cuffs attached to a flexible drive shaft. The drive shaft is placed over a preformed shaped guidewire so that the drive shaft and burrs conform to the shape and profile of the guide wire, i.e., a gentle “S” or “cork-screw” shape. Wulfman, however, requires the preformed guidewire to achieve the non-linear shaping of the drive shaft, a deformed shaping state that is, therefore, present when the device is not rotated. Thus, Wulfman's burrs comprise a sweeping diameter that is limited to, and by, the guidewire shaping. In addition, each of Wulfman's burrs are elliptical and symmetric about the rotational axis of the drive shaft with each center of mass for the burrs being on the drive shaft's rotational axis. Thus, the sweeping diameter of Wulfman is not induced by rotational speed and, therefore, cannot be controlled other than by the guidewire's shaping. Difficulties in positioning the shaped, undeformed, guidewire without damaging the patient's vasculature are also present.

In many of the above-described devices, the mentioned burrs may be secured to the drive shaft with a slip fit that engages the drive shaft relatively loosely if at all. The burrs may then be otherwise secured to the drive shaft with brazing or soldering, for example, which can be inconsistent. The present invention helps to overcome these inconsistencies.

### BRIEF SUMMARY OF THE INVENTION

In one embodiment, a method for attaching a head to a drive shaft may include winding a wire to create a drive shaft with an outside diameter and selecting a head having a length and having a bore with an inside diameter smaller than the outside diameter of the drive shaft. The method may also include forming a tapered tip on a distal end of the drive shaft, the tapered tip having a length greater than the length of the head, and placing the head on the tapered tip such that a distal end of the tapered tip extends from a distal end of the head. The method may also include engaging the distal end of the tapered tip and turning down the drive shaft to create a turned down portion having a length equal to or greater than the length of the head and having a reduced outside diameter that is less than or equal to the inside diameter of the bore. The method may also include advancing the head proximally over the turned down portion. The method may also include disengaging the distal end of the tapered tip, wherein, at or before disengaging, the turned down portion tends toward returning the reduced outside diameter to the outside diameter and is confined from doing so by the bore of the head thereby creating a friction fit between the drive shaft and the head.

In another embodiment, a method for attaching a head to a drive shaft may include selecting a head having a length and having a drive shaft engagement feature with a cross-sectional dimension smaller than an outside diameter of the drive shaft. The method may also include placing the head on an extension feature of the drive shaft such that a distal end of the extension feature extends from a distal end of the head. The method may also include engaging the extension feature and turning down the drive shaft to create a turned down portion having a reduced outside diameter that is less than or equal to the inside diameter of the engagement feature. The method may also include advancing the head proximally over the turned down portion and disengaging the distal end of the extension feature.

In yet another embodiment, a method for attaching a head to a drive shaft may include selecting a head having a length and having a drive shaft engagement feature with a cross-sectional dimension smaller than an outside diameter of the drive shaft. The method may also include reducing the outside diameter of the drive shaft for a portion of the drive shaft less than the full length of the drive shaft. The method may also include placing the head on the portion with the reduced outside diameter.

The figures and the detailed description which follow more particularly exemplify these and other embodiments of the invention.

### BRIEF DESCRIPTION OF THE DRAWINGS

The invention may be more completely understood in consideration of the following detailed description of various embodiments of the invention in connection with the accompanying drawings, which are as follows.

FIG. 1 is a perspective view of an atherectomy device, according to some embodiments;

FIG. 2 is a side and partial cutaway view of the atherectomy device of FIG. 1;

FIG. 3 is a close-up view of a head adjacent a distal end of a drive shaft, according to some embodiments.

FIG. 4 is a close-up view of the head and distal end of the drive shaft of FIG. 3, with the head installed on the drive shaft.

FIG. 5A is a close-up view of a head arranged on a tapered tip of a drive shaft, according to some embodiments.

FIG. 5B is a close-up view of a the head and tapered tip of FIG. 5A with a portion of the drive shaft having been turned down.

FIG. 5C is a close-up view of the head and drive shaft of FIG. 5A with the head in place on the drive shaft and the tapered tip having been trimmed off.

FIG. 6A is a close-up view of a head adjacent a ground down distal end of a drive shaft.

FIG. 6B is a close-up view of the head and distal end of FIG. 6A with the head in place on the distal end of the drive shaft.

FIG. 7A is a close-up view of a head arranged on a tightly wound extension of a drive shaft, according to some embodiments.

FIG. 7B is a close-up view of the head and drive shaft of 7A with a portion of the drive shaft having been turned down.

FIG. 8 is a side view of a tool for use in turning down a distal end of a drive shaft, according to some embodiments.

FIG. 9 is a side view of another tool for use in turning down a distal end of a drive shaft, according to some embodiments.

FIG. 10 is a perspective view of a mandrel for use in turning down a distal end of a drive shaft, according to some embodiments.

FIG. 11 is a perspective view of a drive shaft and a head adapted for engaging the drive shaft from the side, according to some embodiments.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

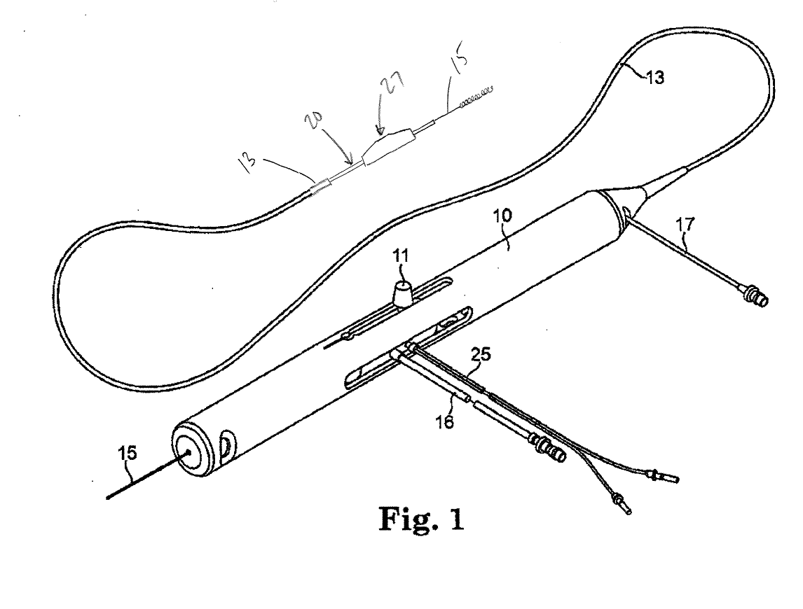

FIG. 1 illustrates one embodiment of a rotational atherectomy device. As shown, the device may include a handle portion 10, an elongated, flexible drive shaft 20 having a head 27 in the form of a burr, crown, or bit, and an elongated catheter 13 extending distally from the handle portion 10. The drive shaft 20 may be constructed from helically coiled wire as is known in the art and the head 27 may be attached thereto. The catheter 13 may include a lumen in which most of the length of the drive shaft 20 is disposed, except for the head 27 and, in some cases, a short section distal to the head 27. The drive shaft 20 also contains an inner lumen, permitting the drive shaft 20 to be advanced and rotated over a guide wire 15. A fluid supply line 17 may be provided for introducing a cooling and lubricating solution (typically saline or another biocompatible fluid) into the catheter 13.

The handle 10 may include a turbine (or similar rotational drive mechanism) for rotating the drive shaft 20 at high speeds. The handle 10 typically may be connected to a power source, such as compressed air delivered through a tube 16 or electrical power delivered with an electrical connection. A pair of fiber optic cables 25, or a single fiber optic cable, for example, may also be provided for monitoring the speed of rotation of the turbine and drive shaft 20. Details regarding such handles and associated instrumentation are well known in the industry. The handle 10 also may include a control knob 11 for advancing and retracting the turbine and drive shaft 20 with respect to the catheter 13 and the body of the handle.

As will be appreciated, there are several different types of heads 27 that may be provided for use in an atherectomy process. In some embodiments, a relatively symmetrical head may be used and in other embodiments an asymmetric head 27 may be provided such that a rotational eccentricity may be provided to increase the diameter of the path that the head 27 travels when rotated. In some embodiments, the head 27 may include a peripheral abrading surface for purposes of increasing the head's ability to clear away stenotic material. In any case, the head 27 may include a drive shaft 20 engagement feature such as a longitudinal bore or other receiving area for attaching the head 27 to the drive shaft 20.

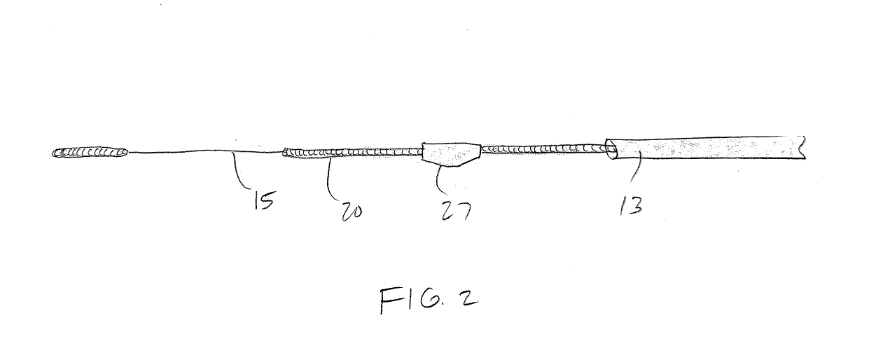

In some cases, the head 27 may include a bore 30 having a diameter that is the same as or just slightly smaller than the outer diameter of the drive shaft 20 and the head 27 may be slip-fit onto the drive shaft. However, it is advantageous to have a fit that is more snug than the slip fit described. A more snug fit, or friction fit, may be provided by providing a head 27 with a bore diameter smaller than the outer diameter of the drive shaft 20. As shown by comparing FIG. 3 to FIG. 4, a friction fit may be provided by elastically reducing the diameter of the drive shaft 20 at a location where the head 27 is to be placed, placing the head 27, and releasing the drive shaft 20 so as to allow it to tend back toward its non-deformed shape. The confining geometry of the head 27 may create a friction fit between the portion of the drive shaft 20 within the head 27 because the drive shaft 20 may be tending toward or biased toward the larger diameter causing the portion of the drive shaft 20 within the head 27 to press radially outwardly against the internal wall of the bore 30, for example. Depending on the type of head 27 that is provided and the details of the drive shaft 20, several techniques may be used to secure the head 27 to the drive shaft 20. In some embodiments, the drive shaft 20 may be turned down, which is to say that the drive shaft 20 may be twisted about its longitudinal axis 32 in a direction causing the coil to tighten, thereby reducing the diameter. That is, for example, in FIG. 3, if the right portion of the drive shaft 20 is held stationary, while the left portion of the drive shaft 20 is twisted clockwise when viewed from the left, the coil may tighten and the diameter may be reduced. As can be appreciated from a review of FIGS. 3 and 4, it can be difficult to engage the distal end of the drive shaft 20 so as to perform the turning down process, while also placing the head 27. That is, if the distal end of the drive shaft 20 is engaged with a tool, for example, the tool may be in the way of placing the head 27. Accordingly, several approaches to solving this problem are provided herein.

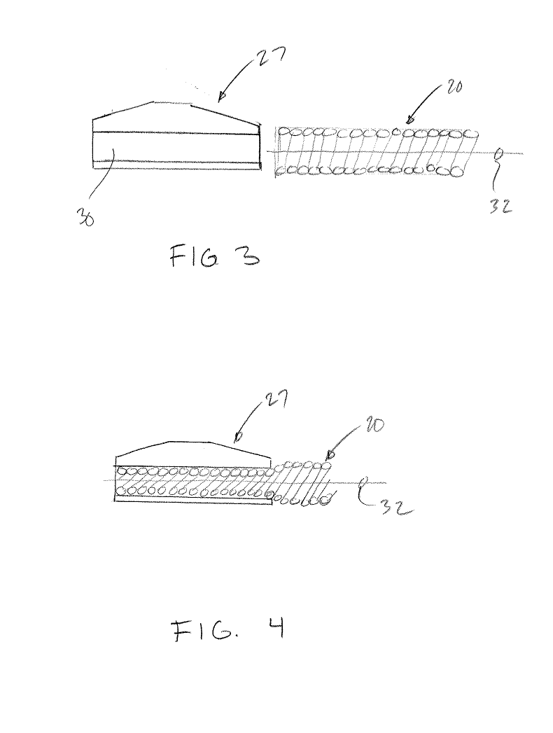

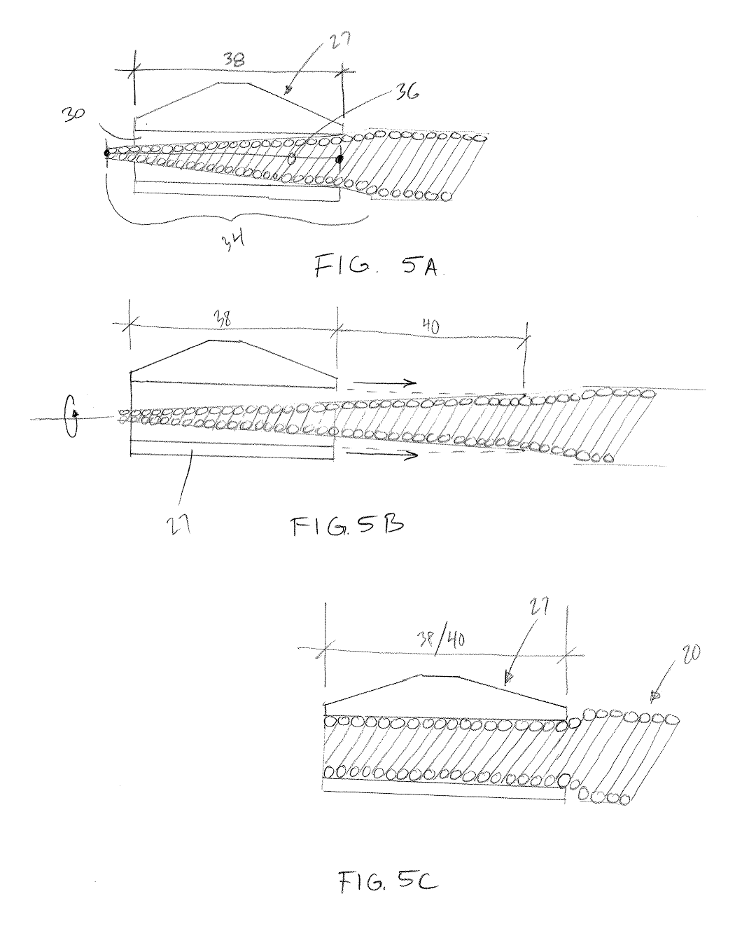

Referring to FIGS. 5A-5C, one embodiment of installing the head 27 is shown. In this embodiment, the drive shaft 20 may be provided with a tapered distal tip 34. The tapered distal tip 34 may have a gradually decreasing diameter as the tip extends distally. At a point along the tapered distal tip 34, the diameter of the tapered tip 34 may be approximately equal to the inner diameter or other cross-sectional dimension of the receiving feature 30, or bore, of the head 27. As the tapered tip 34 extends further distal, the diameter of the tip 34 may continue to decrease at a selected rate. The rate of taper may be selected such that the length 36 of the tapered tip 34, from the point where the diameter of the tip 34 is equal to the inner diameter of the bore 30, is at least slightly longer than the length 38 of the head 27. As such, and as shown in FIG. 5A, the head 27 may be placed on the tapered tip 34 and a portion of the tapered tip 34 may extend out the distal end of the head 27 when the head 27 encounters the tapered tip 34 diameter stopping it from moving further proximally.

The geometry of the tapered tip 34 may therefore be selected to allow the distal tip to project from the distal end of the head 27 when the head 27 is placed on the tapered tip 34. This geometry may allow the distal tip of the taper 34 to be engaged by a turning tool, such that the drive shaft 20 may be turned down and the head 27 may be moved proximally as shown in FIG. 5B without interfering with the turning tool. That is, as shown in FIG. 5B, the turning tool may engage the distal tip of the taper 34 and may turn the tip clockwise when viewed from the left and, assuming that a proximal portion of the drive shaft 20 is held stationary, the turning of the distal tip may cause a length of the drive shaft to be turned down as shown in FIG. 5B. The drive shaft 20 may be turned down by continuing to rotate the distal tip until a length of the drive shaft sufficient to secure the head 27 has a reduced diameter. For example, in one embodiment, a length 40 of the drive shaft proximal to the tapered tip 34 may be turned down and the length 40 of that portion may be approximately equal to the length 38 of the head 27.

Once a sufficient length of the drive shaft 20 has had its diameter reduced, the head 27 may be moved proximally over the turned down portion. Once the head 27 has been placed as desired, the distal tip of the drive shaft 20 may be released, or alternatively unwound, thereby causing the portion of the drive shaft within the head 27 to attempt to return to its original size. As will be appreciated, the inner diameter, or other internal cross-sectional dimension, of the head 27 will confine the drive shaft 20 to a smaller diameter than its at rest diameter causing the drive shaft 20 to press outwardly on the inner surface of the head 27 creating a friction fit. Once the head 27 is in place, the tapered tip 34 may be clipped off, or otherwise removed allowing the inner lumen of the drive shaft 20 to be available for passing through of a guidewire, for example.

In addition to the friction fit provided, the head 27 may also be secured to the drive shaft 20 with a secondary treatment. For example, in some embodiments, the head 27 may also be brazed to the drive shaft. In other embodiments, the head 27 may be welded, soldered, adhered, or otherwise affixed to the drive shaft 20. Still other secondary treatments may be provided for securing the head 27 to the drive shaft 20.

The tapered tip 34 may be pre-treated to allow the handling of the tip as described. That is, in some embodiments the tapered tip 34 may be heat-treated to harden the tip 34 and resist deformations when the tip 34 is gripped or otherwise engaged for the turning down process. In other embodiments, the tapered tip 34 may be brazed, welded, filled, or otherwise treated to resist collapse or deformation under the gripping or other engaging forces used during the turning down process. However, it is to be appreciated that where brazing, welding, filling, or other techniques are used, such techniques may be limited to portions of the tapered tip 34 that have diameters less than the inner diameter of the head 27 so as to avoid a situation where a portion of the tapered tip 34 is both larger than the inner diameter of the head 27 and also resists reduction in size wherein the head 27 may be more difficult or even prevented from being properly positioned.

It is to be appreciated that the turning down of the drive shaft 20 may be performed elastically. That is, the amount of reduction in the diameter may be such that the bending caused in the windings of the drive shaft 20 are limited to elastic bending forces such that, when released, the drive shaft 20 will tend toward returning to its previous size and shape. It is also to be appreciated that, due to the friction between the windings of the drive shaft 20, the effect of the turning down of the distal tip on the diameter of the drive shaft 20 may be reduced as you move proximally along the drive shaft 20. (i.e., away from point where the turning is imparted) This effect may be adjusted or modified depending on how much of, and at what location, the proximal portion of the drive shaft 20 is held stationary.

It is also to be appreciated that while the head 27 is shown to be positioned immediately proximal to the distal end of the drive shaft 20, the drive shaft 20 may be turned down for a longer length in FIG. 5B, allowing the head 27 to be slid further proximally along the drive shaft 20 leaving a longer length of the drive shaft 20 distal to the head 27. Still further, in some embodiments, additional heads 27 may also be installed. In some embodiments a first head 27A may be installed and brazed, soldered, welded, or otherwise secured in place. After installation of the first head 27A, a second head 27B may be installed by turning down the portion of the shaft 20 that is distal to the first head 27A. The second head 27B may then also be brazed soldered, welded, or otherwise secured. In other embodiments, the drive shaft 20 may be turned down and each head 27A, 27B may be properly positioned before any brazing, soldering, welding, or other securing process is performed. In some embodiments, the length of the tapered tip 34 on the drive shaft 20 may be sufficient to allow multiple heads 27 to be placed on the tapered tip 34 such that once the drive shaft 20 is turned down, each of the multiple heads 27A, 27B may be positioned as desired without having to disengage the turning tool to insert an additional head 27.

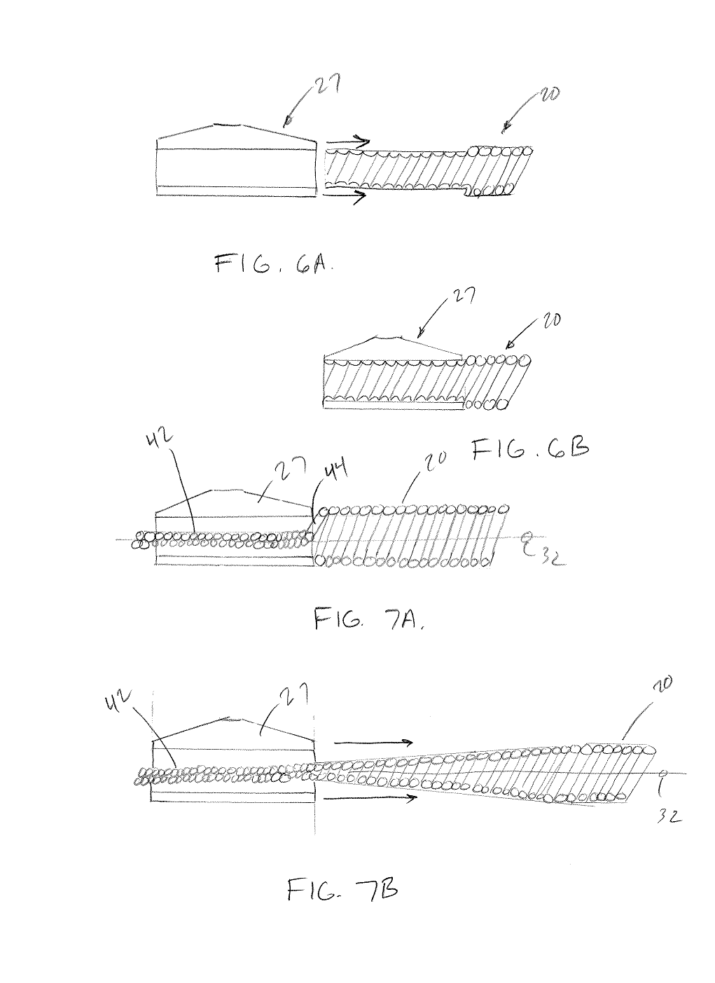

Turning now to FIGS. 6A-6B, a different process for seating a head 27 on a drive shaft 20 is shown. In this embodiment, the diameter of the drive shaft 20 is such that the outer diameter of the drive shaft 20 is larger than the inner diameter of the head 27, but the inner diameter of the drive shaft 20 is smaller than the inner diameter of the head 27. As such, when a portion of the drive shaft 20 is ground away, the drive shaft 20 may be provided with an outer diameter that is the same or similar to the inner diameter of the head 27 allowing the head 27 to be slipped onto the drive shaft 20 and secured.

As with the embodiment of FIGS. 5A-5B, it is to be appreciated that while the head 27 is shown to be positioned immediately proximal to the distal end of the drive shaft 20, the head 27 may be positioned further proximally along the drive shaft 20 leaving a longer length of the drive shaft 20 distal to the head 27. In this embodiment, a longer length of the drive shaft 20 may be ground down. However, in other embodiments, only the portion of the drive shaft 20 where the head is to be seated may be ground down and the portion of the drive shaft distal to the head location may be turned down to allow the head 27 to be slid along the drive shaft 20 to the ground down location. As such, a combination of the turning down approaches as well as the grinding approach may be employed. Still further, in some embodiments, additional heads 27 may also be installed.

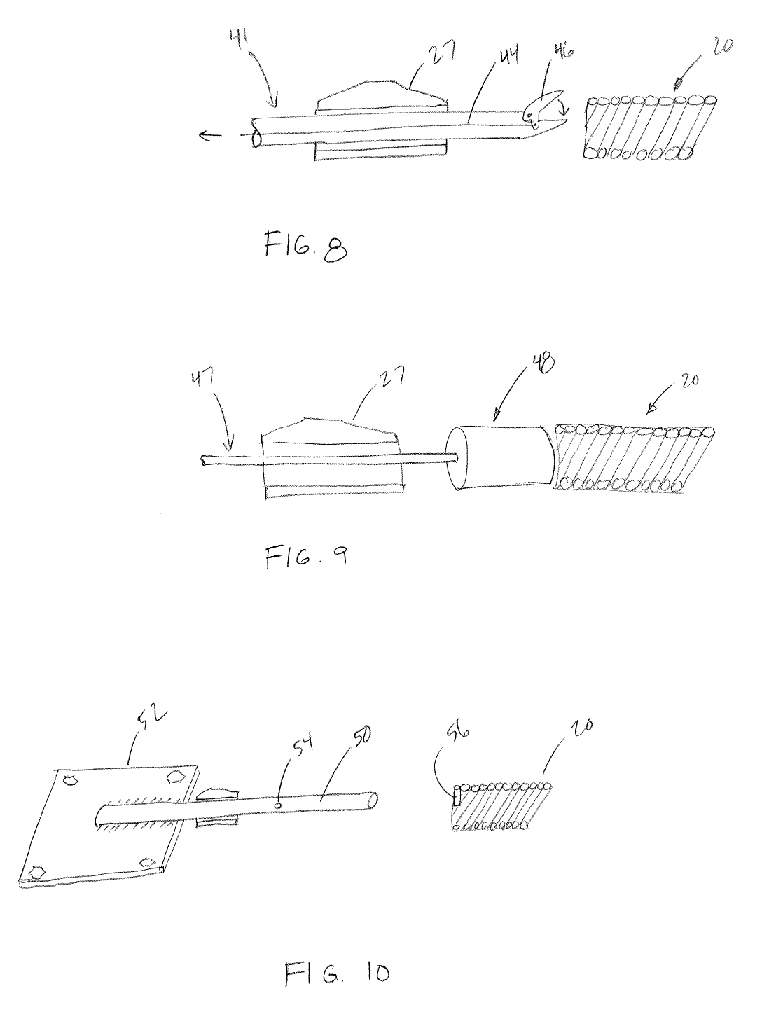

Turning now to FIGS. 7A-7B, an additional embodiment is shown for turning down the drive shaft 20 to allow placement of the head 27. In this embodiment, the distal tip of the drive shaft 20 includes a tightly wound extension 42 extending from its distal end. The tightly wound extension 42 may provide a rotational resistance higher than that of the drive shaft 20 because of its tightly wound geometry. Accordingly, as shown in FIG. 7B, the tightly wound extension 42 may be used by gripping the extension 42 distal to the head 27, turning down a length of the drive shaft 20, and allowing the head 27 to be slipped proximally to a selected location. Upon release or unwinding of the extension 42, the drive shaft 20 may engage the inner bore 30 of the head 27 with a friction fit. The head 27 may then be secured with brazing, welding, soldering, or other securing means.

It is to be appreciated that while the tightly wound extension 42 is shown to be arranged along the longitudinal axis 32 of the drive shaft 20, the extension 42 may also extend parallel to the longitudinal axis 32, but in line with an outer periphery of the drive shaft 20. This may avoid encountering bending forces that may be too high for the arm 44 shown in FIG. 7A that extends from the periphery inwardly to the longitudinal axis. Where the extension 42 is provided in line with the outer periphery of the shaft 20, as the turning down process is performed, the extension may be forced into closer alignment with the longitudinal as shown in FIG. 7B.

Like the tapered tip 34, the tightly wound extension 42 may have a length at least slightly longer than the head 27 such that the distal end of the tightly wound extension 42 may be engaged by a gripping tool or other engaging too while the head 27 is in place thereon. In other embodiments, as also mentioned with respect to the tapered tip 34, the length of the tightly wound extension 42 may be selected to allow one or more heads 27 to be placed thereon such that one step of turning down the drive shaft 20 may allow placement of the one or more heads 27. In addition, and again like the tapered tip 34, all or a portion of the tightly wound extension 42 may be heat treated, brazed, soldered, or welded to provide a stronger member capable of withstanding the gripping or other engaging pressures encountered from the gripping or engaging tool.

It is to be appreciated that the methods shown in FIGS. 5A-5C and 7A-7B involve drive shaft geometries that extend through the head 27 allowing the distal tip of the drive shaft 20 to be gripped or engaged such that the turning down method may be performed. However, an alternative approach may include providing a drive shaft 20 without particularly adapted features and, instead, providing a tool or tools that may be inserted through the head 27 and may be adapted to grip or otherwise engage the distal end of the drive shaft 20, turn it down, and allow the head 27 to be positioned thereon.

FIG. 8 shows a gripping tool 41 that may be used to extend through the head 27, grip the distal tip of the drive shaft 20 (e.g., the distal winding) and turn down the drive shaft 20. The tool 41 may be relatively long and slender and may have an actuation element 44 extending along its length for actuating a jaw or jaws 46 that may be used to grip the drive shaft 20. The tool 41 may include an actuation handle for pulling on the actuation element 44 and the handle may include a pistol type grip, a pliers type grip, or a rotational actuation may be used such that the initial rotation may cause the jaws 46 to engage the drive shaft 20 and further rotation may cause the drive shaft 20 to be turned down.

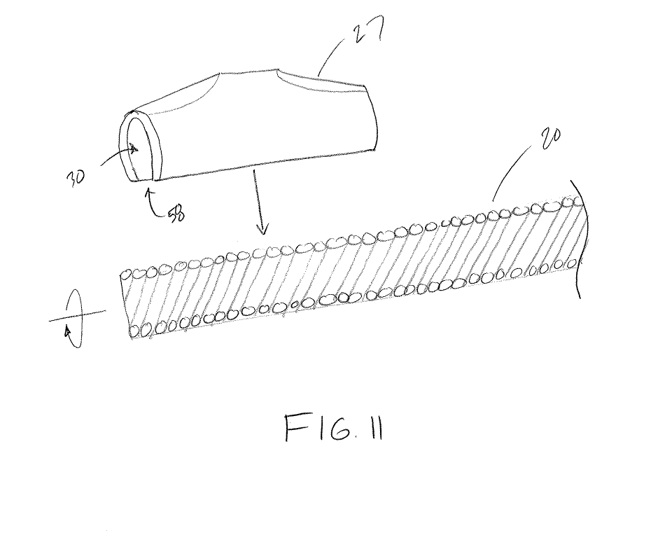

FIG. 9 shows still another tool 47 that may be used to turn down the drive shaft 20. In this embodiment, the tool 47 may include a resilient plug 48 that is slightly larger than the internal diameter of the drive shaft 20 in its uncompressed condition. As such, the plug 48 may be forced into the distal end of the drive shaft 20 thereby frictionally engaging the inner surface of the drive shaft 20. The tool 47 may then be rotated causing the drive shaft 20 to rotate and turn down thereby increasing the frictional engagement of the plug 48 with the drive shaft 20 and also compressing the plug 48 of the tool 47. A head 27 may be positioned on the shaft 20 of the tool 47 by either having forced the plug 48 of the tool 47 through the head 27 or by backing the shaft of the tool 47 through the head 27 and engaging the shaft with a handle or turning tool, for example. Once the drive shaft 20 is turned down sufficiently, the head 27 may be moved proximally onto the drive shaft 20. The positioned of the head 27 may be proximal to the resilient plug 48 such that once the head 27 is placed, the tool 47 may be released or unwound thereby releasing the engagement of the drive shaft 20 with the resilient plug 48. If the excess portion of the drive shaft 20 distal to the head 27 is not desired, the excess portion may be trimmed off.

In addition to particular drive shaft adaptations and particular tools that may be used to “reach” through the head 27, another approach shown in FIG. 10 may be to use a mandrel 50 with a diameter adapted for installing the heads 27. As shown, the mandrel 50 may be supported off of a stationary base 52. Alternatively, the mandrel 50 may held in a user's hand. The mandrel 50 may have a length suitable for placement of one or more heads 27 poised for installation on a drive shaft 20. The mandrel 50 may have an outside diameter less than the inner diameter of the heads 27 and allowing for the thickness of the windings of the drive shaft 20. In addition, adjacent the area for storing heads 27, the mandrel 50 may include an engagement feature 54 for engaging the distal end of the drive shaft 20. In some embodiments, for example, the mandrel 50 may include a hole with a diameter slightly larger than the thickness/diameter of the windings of the drive shaft 20. Other engagement features 54 for engaging the end winding of the drive shaft 20 may also be provided. The distal tip of the drive shaft 20 may have a length 56 of the final winding bent radially inward toward the longitudinal axis of the drive shaft 20. The drive shaft 20 may, thus, be placed onto the mandrel 50 and the bent portion 56 of the final winding may be placed in the hole in the mandrel 50 thereby securing the distal end of the drive shaft 20 against rotation. The remaining portion of the drive shaft 20 may thus be rotated to turn down the drive shaft 20 allowing the heads 27 in the storage area of the mandrel 50 to be moved into place on the drive shaft 20. As with the other embodiments, multiple heads 27 may be placed and each may be secured with brazing, welding, soldering, adhering, or other securing means.

In still other embodiments, a more conventional drive shaft 20 may be provided and more conventional tools may be used to grip and/or engage the drive shaft 20 such that it may be turned down. In this embodiment, as shown in FIG. 11, the head 27 may be provided with a longitudinally extending slot 58 having a width approximately as wide as the bore 30 diameter. In this embodiment, the drive shaft 20 may be turned down to reduce its diameter and the head 27 may be installed from the side, as shown. Like the other embodiments, the head 27 may be brazed, soldered, welded, or otherwise secured to the drive shaft 20 one it is in place and frictionally engaged.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A method for attaching a head to a drive shaft, comprising:
winding a wire to create a drive shaft with an outside diameter;
selecting a head having a length and having a bore with an inside diameter smaller than the outside diameter of the drive shaft;
forming a tapered tip on a distal end of the drive shaft, the tapered tip having a length greater than the length of the head;
placing the head on the tapered tip such that a distal end of the tapered tip extends from a distal end of the head;
engaging the distal end of the tapered tip and turning down the drive shaft to create a turned down portion having a length equal to or greater than the length of the head and having a reduced outside diameter that is less than or equal to the inside diameter of the bore;
advancing the head proximally over the turned down portion; and
disengaging the distal end of the tapered tip, wherein, at or before disengaging, the turned down portion tends toward returning the reduced outside diameter to the outside diameter and is confined from doing so by the bore of the head thereby creating a friction fit between the drive shaft and the head.

2. The method of claim 1, further comprising removing the tapered tip.

3. The method of claim 1, wherein forming a tapered tip further comprises heat treating the tapered tip.

4. The method of claim 1, further comprising applying a secondary means of securing the head to the drive shaft.

5. The method of claim 4, wherein the secondary means comprises one of brazing, welding, and soldering.

6. The method of claim 1, further comprising controllably unwinding the turned down portion to engage the head.

7. A method for attaching a head to a drive shaft, comprising:
selecting a head having a length and having a drive shaft engagement feature with a cross-sectional dimension smaller than an outside diameter of the drive shaft;
placing the head on the an extension feature of the drive shaft such that a distal end of the extension feature extends from a distal end of the head;
engaging the extension feature and turning down the drive shaft to create a turned down portion having a reduced outside diameter that is less than or equal to the inside diameter of the engagement feature;
advancing the head proximally over the turned down portion; and
disengaging the distal end of the extension feature.

8. The method of claim 7, wherein the extension feature is a tapered tip.

9. The method of claim 7, wherein the extension feature is a tightly wound extension.

10. The method of claim 7, further comprising winding a wire to create the drive shaft.

11. The method of claim 10, further comprising forming the extension feature on a distal end of the drive shaft.

12. The method of claim 7, further comprising applying a secondary means of securing the head to the drive shaft.

13. The method of claim 12, wherein the secondary means comprises one of brazing, welding, and soldering.

14. The method of claim 7, wherein the head is an asymmetric head.

15. The method of claim 7, wherein the head is an abrading head.

16. A method for attaching a head to a drive shaft, comprising:
selecting a head having a length and having a drive shaft engagement feature with a cross-sectional dimension smaller than an outside diameter of the drive shaft;
reducing the outside diameter of the drive shaft for a portion of the drive shaft less than the full length of the drive shaft; and
placing the head on the portion with the reduced outside diameter.

17. The method of claim 16, wherein reducing the outside diameter comprises grinding the surface of the drive shaft over a length less than the full length of the drive shaft.

18. The method of claim 16, wherein reducing the outside diameter comprises turning down the diameter of the drive shaft over a length less than the full length of the drive shaft.

19. The method of claim 16, wherein reducing the outside diameter of the drive shaft comprises a combination of grinding and turning down the diameter of the drive shaft.

20. The method of claim 16, further comprising one of brazing, welding, and soldering the head to the drive shaft.
